(19) United States
(12) Patent Application Publication
(10) Pub. No.: US 2020/0163797 A1
(43) Pub. Date: May 28, 2020

(54) OCULAR SURFACE PAIN MITIGATION METHODS AND DEVICES FOR PRACTICING THE SAME

(71) Applicant: THE REGENTS OF THE UNIVERSITY OF MICHIGAN, Ann Arbor, MI (US)

(72) Inventors: Cagri Giray BESIRLI, Ann Arbor, MI (US); Kevin P. PIPE, Ann Arbor, MI (US)

(21) Appl. No.: 16/632,650

(22) PCT Filed: Jul. 5, 2018
(86) PCT No.: PCT/US2018/040883

§ 371 Date: Jan. 21, 2020

Related U.S. Application Data
(60) Provisional application No. 62/537,977, filed on Jul. 28, 2017.

(51) Int. Cl. A61F 9/007; A61M 19/00; A61M 5/42; A61M 5/44

(52) U.S. Cl. CPC A61F 9/007; A61M 19/00; A61M 5/42; A61M 5/44; A61M 2210/0612; A61M 2205/3606; A61M 2205/3673; A61M 2205/8206; A61M 2205/3368

(57) ABSTRACT

Methods of producing anesthesia or analgesia at an ocular tissue site are provided. Aspects of the methods include contacting the ocular tissue site with a cooling element having a temperature ranging from 0 to −30° C., such as 5 to −20° C. for an application time of 30 seconds or less so as to produce anesthesia or analgesia at the target tissue site. Also provided are devices that find use in practicing the methods.

### CROSS-REFERENCE TO RELATED APPLICATIONS

This application claims the benefit and priority of U.S. Provisional Patent Application Ser. No. 62/537,977 filed Jul. 28, 2017; the disclosure of which is herein incorporated by reference.

### INTRODUCTION

Pain is a major limiting factor in many common procedures performed in the inpatient and ambulatory care settings. A very abbreviated list of such procedures includes skin biopsy, fine needle aspiration biopsy, IV insertion, vaccination, injections (including injection of anesthetics and gases), blood draws, central line placements, and finger and heel pricks for blood analysis (glucose measurement). Pharmacologic anesthesia is a primary method of pain reduction, but the delivery of local pharmacologic anesthesia usually requires a painful injection.

The ocular surface is a tissue surface to which, or through which, therapeutic agents may be parenterally delivered. The ability to deliver medication directly into the eye via intravitreal injection therapy (IVT) has transformed the treatment landscape of a number of previously blinding diseases, including macular degeneration and diabetic retinopathy. The success of these therapies in preventing blindness has resulted in a dramatic increase in the number of intravitreal injections performed, with an estimated 4.1 million injections given in the United States alone in 2013. The number of indications for IVT continues to expand, increasing utilization of this therapy significantly every year. The primary limitations of IVT are patient discomfort, ocular surface bleeding, corneal toxicity, and the time constraints of treating the vast number of patients requiring this therapy. These drawbacks relate to current methods of delivering ocular anesthesia to the highly vascularized ocular surface.

To give an ocular injection, the physician first provides ocular surface anesthesia by one or more of a number of methods. These methods involve sodium channel blockers, such as lidocaine and its derivatives, including the following: topical application of anesthetic drops; a subconjunctival injection of lidocaine; placement of cotton tipped applicators (commonly called a “pledget”) soaked in lidocaine over the planned injection site, application of topical lidocaine anesthetic gel, or some combination of these. With pharmacological anesthesia, because the anesthetic agent must diffuse through the various tissue layers to provide nerve blockade, time (at a minimum 30 seconds, but often between 3-10 minutes) is required to achieve adequate anesthesia. Following ocular anesthesia, the physician or an assistant sterilizes the periocular region by coating it in betadine or a similar antiseptic. Optionally, an eyelid speculum is placed, and the physician marks the location of the injection using calipers that guide placement of the needle. The ocular surface is again sterilized, and the physician gives the injection. Current methods of local anesthesia have unique drawbacks and patients often experience discomfort during and after intraocular injections.

### SUMMARY

Methods of producing anesthesia or analgesia at an ocular tissue site are provided. Aspects of the methods include contacting the ocular tissue site with a cooling element having a temperature ranging from 0 to −30° C., such as −5 to −20° C. for an application time of 30 seconds or less so as to produce anesthesia or analgesia at the target tissue site. Also provided are devices that find use in practicing the methods.

### BRIEF DESCRIPTION OF THE FIGURES

FIGS. 1A and 1B provide pictures of a thermoelectric cooling device according to an embodiment of the invention.

FIGS. 2A to 2C provide various three dimensional representations of various aspects of the device shown in FIGS. 1A and 1B.

FIGS. 3A and 3B provide schematic representations of Joule-Thomson process cooling devices that may be employed in embodiments of the invention.

FIG. 4 provides a schematic representation of an endothermic reaction cooling device that may be employed in embodiments of the invention.

FIG. 5 provides a schematic representation of single-stage vapor compression refrigeration cycle cooling device that may be employed in embodiments of the invention.

FIG. 6A provides a schematic representation of an experimental protocol of a study evaluating the safety and efficacy of a thermoelectric cooling device, as described in the Experimental Section, below. FIGS. 6B to 6D provide graphical results of the study.

### DEFINITIONS

As used herein, the term “tissue” refers to one or more aggregates of cells in a subject (e.g., a living organism, such as a mammal, such as a human) that have a similar function and structure or to a plurality of different types of such aggregates. Tissue may include, for example, organ tissue, muscle tissue (e.g., cardiac muscle; smooth muscle; and/or skeletal muscle), connective tissue, ocular tissue (e.g., conjunctiva, episclera, sclera, tenon capsule, retina, choroid, optic nerve), nervous tissue and/or epithelial tissue.

The term “subject” is used interchangeably in this disclosure with the term “patient”. In certain embodiments, a subject is a “mammal” or “mammalian”, where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In some embodiments, subjects are humans. The term “humans” may include human subjects of both genders and at any stage of development (e.g., fetal, neonates, infant, juvenile, adolescent, adult), where in certain embodiments the human subject is a juvenile, adolescent or adult. While the devices and methods described herein may be applied to perform a procedure on a human subject, it is to be understood that the subject devices and methods may also be carried out to perform a procedure on other subjects (that is, in “non-human subjects”).

In some instances, the devices or portions thereof may be viewed as having a proximal and distal end. The term “proximal” refers to a direction oriented toward the operator during use or a position (e.g., a spatial position) closer to the operator (e.g., further from a subject or tissue thereof) during use (e.g., at a time when a tissue is being cooled by the device). Similarly, the term “distal” refers to a direction oriented away from the operator during use or a position (e.g., a spatial position) further from the operator (e.g., closer to a subject or tissue thereof) during use (e.g., at a time when tissue is being cooled by the device). Accordingly, the phrase “proximal end” refers to that end of the device that is closest to the operator during use, while the phrase “distal end” refers to that end of the device that is most distant to the operator during use.

### DETAILED DESCRIPTION

Methods of producing anesthesia or analgesia at an ocular tissue site are provided. Aspects of the methods include contacting the ocular tissue site with a cooling element having a temperature ranging from 0 to −30° C., such as −5 to −20° C. for an application time of 30 seconds or less so as to produce anesthesia or analgesia at the target tissue site. Also provided are devices that find use in practicing the methods.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Additionally, certain embodiments of the disclosed devices and/or associated methods can be represented by drawings which may be included in this application. Embodiments of the devices and their specific spatial characteristics and/or abilities include those shown or substantially shown in the drawings or which are reasonably inferable from the drawings. Such characteristics include, for example, one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal; distal), and/or numbers (e.g., three surfaces; four surfaces), or any combinations thereof. Such spatial characteristics also include, for example, the lack (e.g., specific absence of) one or more (e.g., one, two, three, four, five, six, seven, eight, nine, or ten, etc.) of: symmetries about a plane (e.g., a cross-sectional plane) or axis (e.g., an axis of symmetry), edges, peripheries, surfaces, specific orientations (e.g., proximal), and/or numbers (e.g., three surfaces), or any combinations thereof.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

As summarized, aspects of the invention include producing anesthesia or analgesia at a target tissue site of a subject. As such, in some instances aspects of the methods include producing anesthesia in an ocular target tissue, by which is meant the methods include producing at least some degree of, if not complete loss of, sensation in the ocular tissue site, e.g., via blockage of all feeling in the ocular tissue site). In some instances, aspects of the methods include producing analgesia in the ocular tissue site, by which is meant that the methods provide relief of pain without total loss of feeling in the ocular tissue site.

In some embodiments, the methods include contacting the ocular tissue site with a cooling element having a temperature ranging from 0 to −30° C., such as −5 to −20° C. for an application time of 30 seconds so as to produce anesthesia or analgesia in the ocular tissue site. Cooling elements are components which are configured to be contacted with an ocular tissue site. In the broadest sense, a cooling element may be a solid, such as solid device or component fabricated from a thermally conductive material, e.g., a metal, or a fluid, such as a liquid or gas.

Where the cooling element is a solid component, the cooling element may have a surface that is configured to touch a surface of an ocular tissue site. In such instances, cooling elements may have a body, e.g., of a solid material, such as a thermally conductive material (e.g., a metal or alloy), that has a distal, ocular surface contacting end. In such instances, the distal ocular surface contacting end may be planar or curved, and may be configured to contact a defined area of ocular surface. While the area of the distal ocular tissue contacting surface may vary, in some instances the area of this surface ranges from 4 mm² to 100 mm², such as 20 mm² to 50 mm². In some instances, one or more protrusions may be present on the distal ocular tissue contacting surface, where in some instance the number ranges from 1 to 4, such as 2 to 3. Such protrusions may serve a variety of functions, including but not limited as providing indentations on the ocular surface to mark where anesthesia/analgesia has been induced by the device, e.g., for subsequent therapeutic agent delivery (e.g., so as to know where to insert a needle). When present, the height of such protrusions may vary, ranging in some instances from 0.1 to 3 mm, such as 0.5 to 2 mm. While the shape of such protrusions may vary, of interest are protrusions that have atraumatic shapes, such as protrusions with rounded tops, etc.

As summarized above, the cooling element has a temperature sufficient to impart anesthesia or analgesia to the ocular tissue surface. In some instances, during use the cooling element has a temperature ranging from 0 to −30° C., such as 5 to −20° C., and in some instances during use the cooling element has a temperature ranging from −7 to −20° C., such as −8 to −15° C., where in some instances the cooling element has a temperature that is −10° C., −7° C. or −5° C. during use. While the application time of 30 seconds or less may vary, in some instances the application time ranges from 5 to 30 seconds, such as 10 to 20 seconds, e.g., 10 seconds.

Any convenient device having a cooling element that functions as described above may be employed in practicing methods of the invention. Specific devices of interest include handheld devices that include a cooling system that include a cooling element, e.g., as described above. In such embodiments, as the devices are handheld, they are configured to be held easily in the hand of an adult human. Accordingly, the devices may have a configuration that is amenable to gripping by the human adult hand. The weight of the devices may vary, and in some instances may range from 0.05 to 3 pounds, such as 0.1 pounds to 1 pound, where in some instances the weight is 1 pound or less, such as 0.5 pounds or less. Handheld devices of the invention may have any convenient configuration, where examples of suitable handle configurations are further provided below.

While the cooling system of such devices may vary, specific cooling systems of interest include, but are not limited to: thermoelectric cooling systems, liquid evaporation cooling systems, solid sublimation cooling systems, solid melting cooling systems, Joule-Thompson cooling systems, thermodynamic cycle cooling systems, endothermic reaction cooling systems and low-temperature substance cooling systems, and the like, where in some instances a given cooling system may include a combination of two or more of the above types of cooling systems.

In some instances, the cooling system is a thermoelectric cooling system, e.g., one that includes one or a combination of thermoelectric (Peltier) devices. While thermoelectric cooling systems employed in embodiments of devices of the invention may vary, in some instances the thermoelectric cooling systems include a cooling element in the form of a cold tip that is configured to contact an ocular tissue site, a power source, a controller, a cooling power concentrator, one or more Peltier unit modules, and a heat sink. It should be understood that, in some embodiments, a given thermoelectric cooling system may include a heating element (not shown) that operates in conjunction with the cooling elements to precisely maintain a desired temperature and/or heat flux. Further details regarding embodiments of thermoelectric cooling systems that may be employed in methods of the invention are provided in U.S. Published Patent Application Publication No. 20160279350; the disclosure of which is herein incorporated by reference.

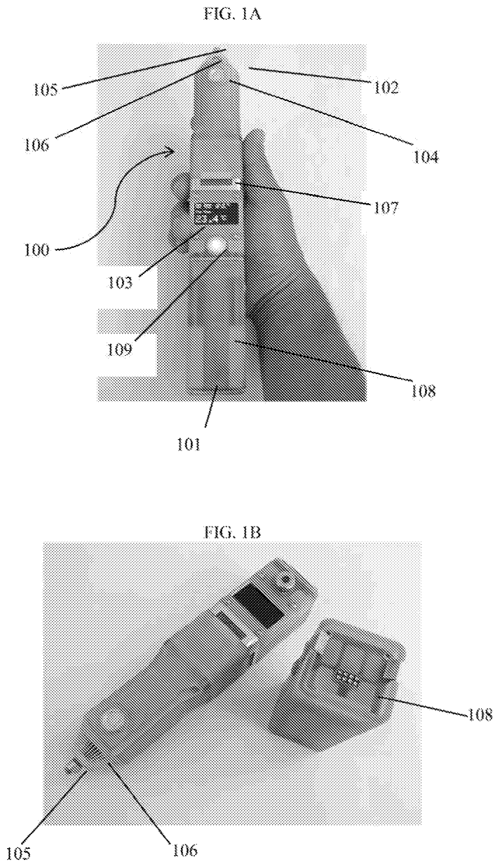

An example of a hand-held thermoelectric cooling system device that may be employed in methods of the invention is shown in FIGS. 1A and 1B. As shown in FIG. 1A, device 100 is a handheld device that includes a proximal end 101 and a distal end 102. Device 100 measures approximately 10 inches in length and 1.5 inches in diameter, and weighs approximately 0.8 pounds. Located at distal end 102 is replaceable cooling element, i.e., cool tip, 105, which has a diameter of 4 mm and a distal surface area of 12.6 mm².

The temperature of this cooling element can be a user-specified temperature setpoint between 0 and −10° C., which can be implemented by activation of button 109. Operation of the button 109 includes: 1) Clockwise rotation to increase the cold tip temperature setpoint, 2) Counter-clockwise rotation to decrease the cold tip temperature setpoint, 3) Clockwise rotation while the button is pushed, to increase the timer duration, 4) Counter-clockwise rotation while the button is pushed, to decrease the timer duration, and 5) Double-pressing to activate the Peltier modules.

A second tactile button 104 located near the grip position is used to activate (or deactivate) the timer. When the timer is activated, the device produces two consecutive beeping sounds at low and high frequencies, followed by beeping sounds every 10 seconds during the timer duration, and finally two long consecutive beeping sounds at high and low frequencies when the timer duration has expired. If the “Timer” button is pushed again before the set time is reached, two short consecutive beeps at high and low frequencies occur and the timer is immediately terminated.

Also shown at the distal end are intake vents 106. In the middle of the device is air vent 107, as well as display 103. Device 100 includes a temperature regulating feedback loop that maintains highly accurate and precise temperature control and an integrated timer that controls the duration of cooling, both of which facilitate sufficient cooling and prohibit excessive cooling. The proximal end of the device includes a rechargeable battery, which may be separated from the remainder of the device as shown in FIG. 1B, e.g., to facilitate recharging. In the device shown in FIGS. 1A and 1B, the rechargeable battery is a rechargeable lithium ion battery pack (28 Wh), which provides sufficient energy on a single charge to operate the device at −10° C. for approximately one hour (−100 treatments), and can be easily removed from the rest of the device to facilitate simple charging.

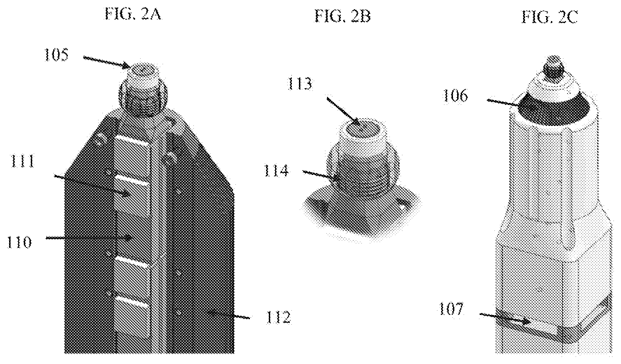

FIGS. 2A to 2C provide different three-dimensional renderings of various components of the device shown in FIGS. 1A and 1B. Device 100 uses thermoelectric (Peltier) cooling to create the low temperature required for cryoanesthesia. As shown in FIG. 2A, the device includes an inner arm 110 (i.e., a cold arm) (which functions as a cooling power concentrator) made from a thermally conductive metal, where inner arm 110 is simultaneously cooled by a group of Peltier modules 111 and is thus able to achieve a large cooling power and maintain a low temperature at the cooling surface on the replaceable cooling tip 105. Heat generated by Peltier modules 111 is rejected to an area of a heat sink shown as fins 112 (which function as a heat sink) that is not only large in surface area and hence efficient but also extended away from the cooling surface 105, thus allowing a narrow shape with better visual clearance during use.

Inlet openings 106 as shown in FIG. 2C allow for air to be drawn in by an electric fan. The air cools the fins 112 and exits through vents 107 at a location far from the replaceable tip (FIG. 2C). The location of the vents 107 far from the replaceable tip 105 not only reduces parasitic convection heating at the tip surface but also prevents the airflow from drying the eye surface. Temperature control is achieved by an electrical feedback loop that samples the tip temperature using a thermal sensor and provides appropriate power to the thermoelectric coolers via pulse width modulation (PWM) of the DC battery source. While the cooling surface is unable to reach a temperature lower than −12° C. when the device is operated at typical room temperature (20° C.), a failsafe automatically powers the device off if the cooling surface falls below −12° C. Other automatic power offs occur if the battery temperature exceeds 60° C. or the heat sink temperature exceeds 50° C.

A single button on the device is used to set the cold tip temperature and timer duration, as well as power the Peltier modules.

During use, a user readies the device by first inserting a replaceable tip into the cold arm and using a vice-like mechanism 114 (such as a compression ring threadedly engaging corresponding threads disposed on an exterior surface) as shown in FIG. 2B to hold it in place, which provides both for convenient tip replacement and sufficient pressure between the replaceable tip and cold arm for good thermal contact. The replaceable tip 105 is coated by a thin hydrophobic polymer layer that mitigates ice adhesion between the device and tissue. Suitable hydrophilic polymer materials for this layer include, but are not limited to: Teflon, Parylene, and the like. Alternatively or in addition to such a polymer layer, during use the tip may warmed prior to removal in a manner sufficient to prevent ice adhesion, e.g., to a temperature of −2° C. Two small protrusions 113 on the replaceable tip leave temporary indentations on the eye surface to guide subsequent treatment (e.g., placement of the intravitreal injection needle 3 or 4 mm from the corneal limbus). After setting the desired cold tip temperature set-point and timer duration and inserting the replaceable tip, the user operates the device by first double-pressing the main (“rotation & push”) button, which turns on the Peltier modules and brings the tip temperature to the set-point (this takes approximately 15 seconds for a set-point of −10° C.). The user then brings the tip into contact with the patient's eye and presses the “timer” button (which responds with a beep). If the timer duration is set to 10 (or 30) seconds, the device maintains −10° C. for 10 (or 30) seconds and then beeps. After the timer duration, the device returns to ambient temperature.

Further details regarding the above described device may be found in U.S. application Ser. No. 15/079,132 published as U.S. 2016/0279350; the disclosure of which is herein incorporated by reference.

Alternatively, devices employed in methods of the invention may include other non-thermoelectric cooling systems. Such non-thermoelectric cooling systems include, but are not limited to: liquid evaporation cooling system, solid sublimation cooling system, Joule-Thompson cooling system, thermodynamic cycle cooling system, an endothermic reaction cooling system and a low-temperature substance cooling system.

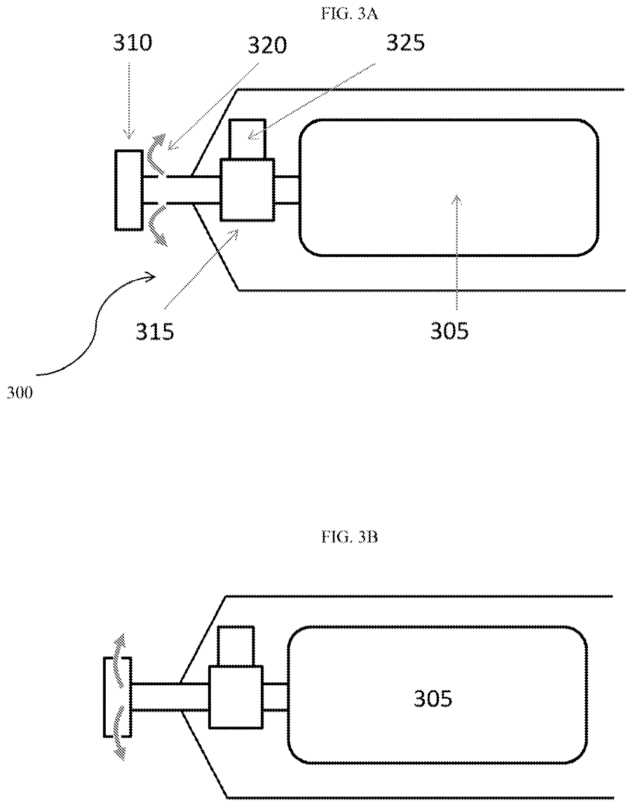

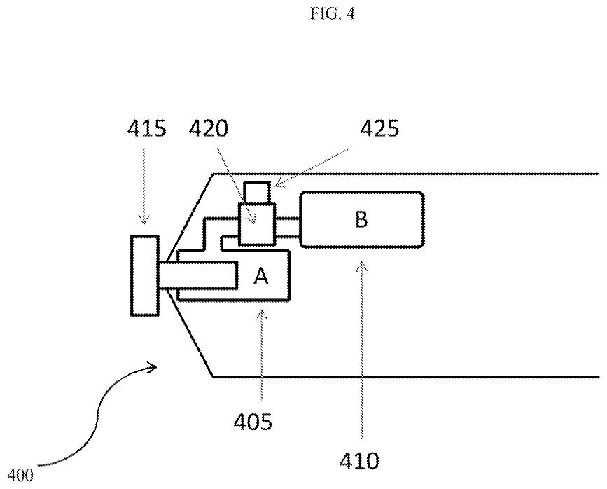

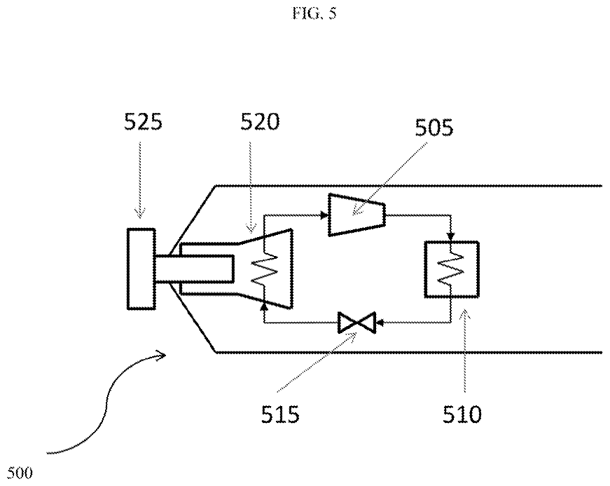

In some instances, devices that include Joule-Thompson cooling systems are employed. In Joule-Thomson cooling devices, a source of pressurized fluid, e.g., a liquid or gas, is controllably released from a source at a location at least proximal to a cooling element, such as a cold tip, in a manner sufficient to provide for the desired temperature of the cooling element. Feedback control of the temperature of the cooling element may be accomplished, for example, by dynamically controlling fluid release, e.g., by dynamically controlling the width of an aperture of a container of the fluid using an electrically actuated valve. An embodiment of such a device is shown in FIG. 3A. As shown in FIG. 3A, device 300 includes a container 305 of pressurized fluid. The device also includes cooling element in the form of a cold tip 310. To provide for a desired temperature of cold tip 310, a valve 315 is opened using actuator 325, and the fluid expands 320 through the valve, producing cooling. The expanding fluid 320 cools the device tip 310. (Alternatively, the expanding fluid 320 may be released in a manner such that it cools the eye directly). The expanding fluid may cool the tip by being incident upon it (e.g., as shown in FIG. 3A), or it may flow through channels within the tip (e.g., as shown in FIG. 3B) or near the tip. It may also be thermally coupled to a heat exchanger or thermally conductive material which then cools the tip.

Cooling devices that may be employed in embodiments of the invention also include endothermic reaction cooling devices. Endothermic reaction cooling devices are devices in which two or more reactants are combined in an endothermic reaction, which reaction draws heat from the device in a manner sufficient to provide a cooling element with a desired temperature, such as described above. An example of such a device is shown in FIG. 4. As illustrated in FIG. 4, device 400 includes a first container 405 that includes a first reactant and a second container 410 that includes a second reactant. To cool the cooling element 415, a valve 420 is opened by actuator 425, which allows for mixing of the reactants of compartments 405 and 410. It is noted that while combination of reactants is mediated by opening valve 420, other approaches to providing for combination of reactants may be employed. For example, a removable barrier, such as a breakable membrane, may be employed, as desired. The resultant endothermic chemical reaction reduces the temperature of the thermally coupled cooling element 415. Feedback control of the cooling power may be accomplished, for example by dynamically controlling the rate of mixing. The mixed substance, i.e., product composition of the endothermic reaction that has a reduced temperature, may cool the device tip 415, or it may cool the eye directly. It may cool the tip 415 by being incident upon it, or it may flow through channels within or near the tip. It may also be thermally coupled to a heat exchanger or thermally conductive material which then cools the tip. Pairs of reactants A and B that may be employed in device 400 may vary, and include but are not limited to: water and ammonium chloride, ammonium nitrate, calcium ammonium nitrate, or urea; or barium hydroxide and ammonium chloride; and the like.

Yet another type of cooling device that may be employed in methods of the invention includes a thermodynamic cycle cooling device. Such devices include a working fluid that completes a thermodynamic cycle to achieve desired cooling of a cooling element. Examples of thermodynamic cycles that may be employed in such devices include Stirling cycles, reverse Brayton cycles, Gifford-McMahon coolers, Joule-Thomson cycles, vapor-compression refrigeration cycles, or pulse tube coolers. An embodiment of such a device is shown in FIG. 5, which figure provides an illustration of a single-stage vapor compression refrigeration cycle cooling device 500. As shown in FIG. 5, device 500 includes a compressor 505, condenser 510, expansion valve 515, and evaporator 520. The evaporator 520 is thermally coupled to the tip 525, for example by a heat exchanger, thermally conductive material, or evaporator channels which flow within or near the tip. Feedback control of the cooling power may be accomplished, for example, by dynamically controlling the flow rate of the working fluid (for flow loop based techniques such as the vapor compression refrigerator) or the operating frequency (for pulsed techniques such as the pulse tube cooler).

Devices that may be employed in methods of the invention also include sensible heat cooling devices, such as low temperature substance cooling devices, which are devices that include a low temperature substance that is used to cool a cooling element of the device. In one embodiment of such devices, a precooled substance is introduced to the device. This cold substance then cools the device tip (or the surface of the eye directly).

Devices that may be employed in methods of the invention also include latent heat cooling devices, where such devices may provide cooling via a phase change, such as may be accomplished via melting, evaporating, sublimating, etc. As such, some instances, the device includes a phase change material. In such embodiments, heat from an ocular surface may be absorbed by the phase change material (latent heat). Control of the cooling temperature may be achieved, for example, by choosing a material with an appropriate phase change (e.g., melting, evaporation, or sublimation) temperature. Examples of phase change materials that may be employed in such devices include: refrigerants (e.g., R-134a), dry ice, and the like. The ocular tissue site that is cooled using methods of the invention, e.g., as described above, may vary. In some instances, the ocular tissue site is a region that begins at the corneal limbus and extends anywhere from 1 mm to 10 mm posterior to the limbus, e.g., 2 mm to over 8 mm posterior to the limbus, such as 3 mm to 6 mm from the corneal limbus, e.g., 3 to 4 mm from the corneal limbus, e.g., to allow intraocular injection, for example via pars plana or pars plicata, where the methods are methods of delivering to ocular tissue, or through ocular tissue to a cavity within the eye, such as the aqueous chamber or vitreous chamber, e.g., as described in greater detail below. Ocular delivery sites may include conjunctiva, episclera, and sclera of the eye. Ocular tissue delivery sites of interest include those that provide for intravitreal injection therapy (IVT), retrobulbar injection therapy, subtenon or subconjunctival injection therapy, subretinal injection therapy, suprachorodial injection, intracameral injection therapy, injection into the ciliary muscle and/or ciliary body, and the like.

Methods of the invention, e.g., as described above, find use in a variety of different applications where it is desired to produce anesthesia or analgesia at an ocular tissue site. Such applications or procedures include, but are not limited to, therapeutic agent delivery applications, biopsy applications, ocular examination applications, cataract removal, ocular surgery and the like. In such procedures, methods as described here may be employed to reduce the magnitude of pain experienced by a subject during the procedure. While the magnitude of pain reduction in such embodiments may vary, in some instances the magnitude of pain reduction is 90% or more, such as 95% or more, e.g., as determined using a visual analog scale (1 to 10, with 10 being severe pain and 1 being no pain) (such as further described in Shah et al., “A novel lidocaine hydrochloride ophthalmic gel for topical ocular anesthesia”, Local and Regional Anesthesia (2010) 3: 57-63). In some embodiments, methods as described here may be employed to reduce the magnitude of pain experienced by a subject following a procedure. While the magnitude of pain reduction in such embodiments may vary, in some instances the magnitude of pain reduction is 90% or more, such as 95% or more, e.g., as determined using a visual analog scale (such as described in Georgakopoulos et al., “Effect of Bromfenac on Pain Related to Intravitreal Injections: A Randomized Crossover Study,” Retina (2017) 37(2):388-395).

In some instances, methods of the invention, e.g., as described above, find use in delivering a therapeutic agent to an ocular tissue site, e.g., where the procedure as described above is a therapeutic agent administration procedure, such as an ocular injection procedure. In some instances, the ocular tissue site is a region that begins at the corneal limbus and extends anywhere from 2 mm to over 8 mm posterior to the limbus, such as 3 mm to 6 mm from the corneal limbus, e.g., 3 to 4 mm from the corneal limbus, e.g., to allow intraocular injection via pars plana or pars plicata. Ocular tissue delivery sites may include conjunctiva, episclera, and sclera of the eye.

In some instances, the subject devices are used to prepare an ocular tissue site for an injection of a therapeutic agent, i.e., an injection therapy. The methods may be used in a variety of different ocular injection therapies, where ocular injection therapies of interest include, but are not limited to: intravitreal injection therapy (IVT), retrobulbar injection therapy, subtenon injection therapy, subretinal injection therapy, suprachoroial injection, subconjunctival injection therapy, intracameral injection therapy, and the like.

Examples of therapeutic agents that may be delivered by such ocular injection therapies include, but are not limited to steroids such as corticosteroids including dexamethasone (e.g., Ozurdex™), fluocinolone (e.g., Retisert™ or lluvien™, loteprednol, difluprednate, fluorometholone, prednisolone, medrysone, triamcinolone, betamethasone and rimexolone; nonsteroidal anti-inflammatory agents such as salicylic-, indole acetic-, aryl acetic-, aryl propionic-and enolic acid derivatives including bromfenac, diclofenac, flurbiprofen, ketorolac tromethamine and nepafenac; antibiotics including azithromycin, bacitracin, besifloxacin, ciprofloxacin, erythromycin, gatifloxacin, gentamicin, levofloxacin, moxifloxacin, ofloxacin, sulfacetamide and tobramycin; VEGF inhibitors such as tyrosine kinase inhibitors, antibodies to VEGF, antibody fragments to VEGF, VEGF binding fusion proteins (e.g., pegaptinib, ranibizumab, bevacizumab, aflibercept, brolucizumab); PDGF inhibitors, antibodies to PDGF, antibody fragments to PDGF, PDGF binding fusion proteins (e.g., (Fovista™); anti-complement agents such as anti-Factor D antibodies (lampalizumab), anti-TNF alpha agents such as antibodies to TNF-alpha, antibody fragments to TNF-alpha and TNF binding fusion proteins including infliximab, etanercept, adalimumab, certolizumab and golimumab; mTOR inhibitors such as sirolimus (Opsiria™), sirolimus analogues, Everolimus, Temsirolimus and mTOR kinase inhibitors; gases such as air, SF6, C2F6, C3F8, and others used in, for example, pneumatic retinopexy; cells such as mesenchymal cells (e.g. mesenchymal stem cells), or cells transfected to produce a therapeutic compound; neuroprotective agents such as antioxidants, calcineurin inhibitors, NOS inhibitors, sigma-1 modulators, AMPA antagonists, calcium channel blockers and histone-deacetylases inhibitors; antihypertensive agents such as prostaglandin analogs, beta blockers, alpha agonists, and carbonic anhydrase inhibitors; aminosterols such as squalamine; antihistamines such as H1-receptor antagonists and histamine H2-receptor antagonists; therapeutic cells; tyrosine kinase inhibitors and nucleic acid based therapeutics such as gene vectors, complement inhibitors; chemotherapeutic agents; insulin; plasmids and siRNA.

The methods and devices of the invention, e.g., as described above, may be employed in conjunction with methods of delivering a therapeutic agent to treat a variety of different disease conditions. Disease conditions of interest include, but are not limited to, ocular conditions, such as ocular disease conditions, such as intraocular neovascular disease conditions. An “intraocular neovascular disease” is a disease characterized by ocular neovascularization. Examples of intraocular neovascular diseases include, for example, proliferative retinopathies, choroidal neovascularization (CNV), age-related macular degeneration (AMD), geographic atrophy (GA), diabetic and other ischemia-related retinopathies, diabetic macular edema, pathological myopia, von Hippel-Lindau disease, histoplasmosis of the eye, Central Retinal Vein Occlusion (CRVO), Branch Retinal Vein Occlusion (BRVO), pterygium, corneal neovascularization, and retinal neovascularization. The term “age-related macular degeneration” refers to a medical condition which usually affects older adults and results in a loss of vision in the center of the visual field (the macula) because of damage to the retina. Some or all of these conditions can be treated by intravitreal injection of a VEGF-antagonist, e.g., as described above. Other ocular conditions that may be treated in accordance with aspects of the invention include, but are not limited to: retinal detachments (pneumatic retinopexy), by using devices of the invention to inject a gas into the eye, where the device may control the depth of injection to a desired/optimal depth. Disease conditions of interest also include central serous chorioretinopathy and uveitis, including anterior uveitis, pars planitis, intermediate uveitis, and posterior uveitis.

The devices may be employed to deliver a therapeutic agent to a target tissue delivery site of different types of subjects. Generally such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In certain embodiments, the subjects are humans. The methods may be diagnostic and/or therapeutic methods.

The following example is offered by way of illustration and not by way of limitation.

### Experimental

### A. Treatment Protocol

A cryoanesthesia cooling device as shown in FIGS. 1A and 1B and described above was employed. The employed cryoanesthesia cooling device was a handheld instrument measuring approximately 10 inches in length and 1.5 inches in diameter. In the protocol, a 4 mm diameter tip of the device that had been cooled to the temperature set-point (e.g., −5° C., −7° C., or −10° C.) via activation of a switch on the device was placed against the eye. Cooling slows conduction of pain fibers in the conjunctiva (outermost layer of the eye), episclera, and the sclera (white of the eye) to a depth less than a millimeter. The key features of the device include a temperature regulating feedback loop to maintain highly accurate temperature control and a lockout mechanism to prevent excessive cooling. Two small protrusions on the tip leave temporary indentations on the surface of the eye to guide subsequent placement of the intravitreal needle. The cooling region of the device is located on a replaceable metal tip. An “ON” switch is first activated to cool the contact region of the device, after which this region is placed against the eye, initiating rapid cooling to anesthetize the injection site while simultaneously marking the cooled location. After the specified cooling time is reached, the device is removed, immediately after which the physician performs intravitreal injection.

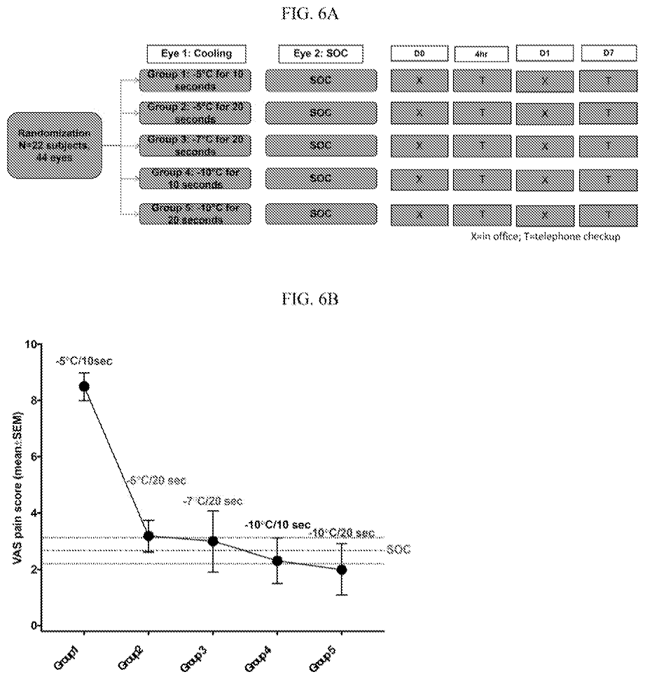

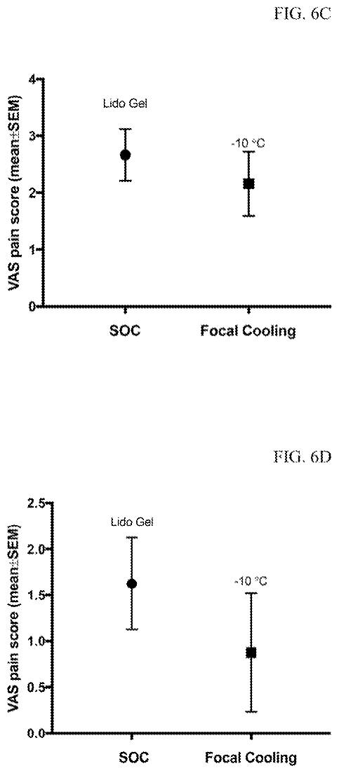

22 patients were treated with focal cooling via cryoanesthesia (Cooling) or standard of care (SOC) lidocaine-based anesthesia according to the schematic shown in FIG. 6A.

For each patient, the brow above the eye to receive intravitreal treatment was marked. Next, one drop of proparacaine was applied into the eye at least 5 minutes before any additional steps were taken. Once the patient was ready, another drop of proparacaine was applied and the physician or his assistant then swabbed the lids with betadine. The physician then placed a lid speculum in the patient's eye. A drop of betadine was applied at the site of treatment. The physician or his assistant then activated the cryoanesthesia device by pressing the main button, following which the LED display was reviewed to verify that the device was set to achieve the correct temperature and time setpoints depending on the arm of the study to which the patient was randomized. The physician then adjusted the temperature and time setpoints using the main device button if they were not correctly entered. The physician then verbally reviewed the selected treatment temperature and time, after which the physician activated the device to enable the device tip to reach the required temperature. Immediately prior to placing the device tip against the eye, the physician pressed the activation switch, after which the physician applied the device to the surface of the eye. When the timer sounded, the physician removed the device from the surface of the eye. The device tip had markers on it that left a temporary indentation 3 and 4 mm from the limbus. Immediately before giving the injection, the physician placed a drop of betadine on the surface of the eye at the site marked for the injection and waited 20 seconds. The physician then gave the intravitreal injection.

The procedure for the patients receiving SOC was similar to that described above. The brow above the eye to receive intravitreal treatment was marked. Next, one drop of proparacaine was applied into the eye at least 5 minutes before any additional steps were taken. Once the patient was ready, another drop of proparacaine was applied and the physician or assistant then swabbed the lids with betadine. 4 patients received 3 sets of cotton tipped pledgets soaked in 4% lidocaine placed over the injection site. Each set of pledgets was kept on the eye for 1 minute. The 3 minute waiting period was needed to allow the lidocaine in the pledgets to diffuse through the sclera. 18 patients received 3.5% lidocaine gel that was placed on the eye and left in place for a minimum of 3 minutes prior to IVT. The 3 minute waiting period was needed to allow the lidocaine gel to diffuse through the sclera. Following completion of numbing with lidocaine, the eyelids were swabbed with betadine and the physician placed the lid speculum. The physician the used a caliper to measure 3 or 4 mm from the limbus of the eye, placed a drop of betadine over this spot, waited 20 seconds and then administered the injection.

### B. Results

22 subjects were randomized to receive focal cooling via cryoanesthesia in one eye and standard ocular anesthesia (SOC) in their other eye, as described above. The eye receiving cryoanesthesia was randomized to receive one of five groups with different temperatures and/or time points of cooling, as outlined in the design schematic above. The other eye received a standard of care (SOC) anesthesia, which included either cotton tipped pledgets soaked in 4% lidocaine (4 patients) or 3.5% lidocaine gel (18 patients).

The primary outcome of this study was patient-rated pain during intravitreal injection (IVT), which was assessed as 1 to 10 using a visual analogue scale (VAS). Subjects were asked to record the amount of pain they experienced during the act of injection. Exploratory outcome included patient-rated pain 4 hours following the injection. In addition, all subjects underwent pre- and post-treatment microscopic ocular examinations at the slit lamp to document subconjunctival hemorrhage, conjunctival hyperemia, conjunctival injection, anterior chamber inflammation, and corneal keratopathy.

No treatment-related adverse events were reported, and no subjects exhibited any signs of ocular toxicity following the use of cryoanesthesia.

Feasibility study (FS) demonstrated a temperature and time dependent treatment response, with higher injection pain scores in group 1 compared to groups 2 to 5 (FIG. 6B). FIG. 6B provides the patient reported intravitreal injection pain measured by visual analog scale (VAS,1-10). As shown in FIG. 6B, focal cooling with decreasing temperatures and increasing time of treatment provides effective pain control during intravitreal injection compared to standard of care (SOC, 3.5% lidocaine gel). The mean pain score of patients in the SOC arm is shown as a dashed line and dotted lines represent +1-standard error of the mean (SEM). Temperature and duration of treatment are shown above data points for each group. Data is represented as a mean +1-SEM. The lidocaine gel control arm in the FS had a mean pain score of 2.9±0.47 on VAS, which is similar to historical data, validating the study design and data collection.

FIG. 6C provides patient reported intravitreal injection pain measured by visual analog scale (VAS, 1-10). Pooled data from the focal cooling arms using −10° C. for 10 or 20 seconds (−10° C.) shows effective pain control during intravitreal injection compared to standard of care (SOC, 3.5% lidocaine gel, Lido gel). Data is represented as mean +1-SEM. Pooled data from Groups 4 and 5 treated with −10° C. showed mean injection VAS pain scores similar to SOC gel (FIG. 6C).

FIG. 6D provides patient reported post-intravitreal injection pain measured by visual analog scale (VAS, 1-10) 4 hours after treatment. Pooled data from the focal cooling arms using −10° C. for 10 or 20 seconds (−10° C.) shows effective post-intravitreal injection pain control compared to standard of care (SOC, 3.5% lidocaine gel, Lido gel). Data is represented as mean +1-SEM. Pooled analysis of Groups 4 and 5 showed that mean 4 hour post-injection VAS pain scores were similar between experimental group treated with −10° C. focal cooling and SOC gel (FIG. 6D).

Notwithstanding the appended claims, the disclosure set forth herein is also defined by the following clauses:
1. A method of producing anesthesia or analgesia at an ocular tissue site of a subject, the method comprising:

contacting the ocular tissue site with a cooling element having a temperature ranging from 0 to −30° C. for an application time of 30 seconds or less so as to produce anesthesia or analgesia at the ocular tissue site.
2. The method according to Clause 1, wherein the cooling element has a temperature ranging from −5 to −20° C.
3. The method according to Clause 1, wherein the cooling element has a temperature of about −10° C.
4. The method according to Clause 1, wherein the cooling element has a temperature of about −7° C.
5. The method according to Clause 1, wherein the cooling element has a temperature of about −5° C.
6. The method according to any of Clauses 1 to 5, wherein the application time ranges from 5 to 30 seconds.
7. The method according to Clause 6, wherein the application time ranges from 10 to 20 seconds.
8. The method according to Clause 1, wherein the cooling element has a temperature of about −10° C. and the application time is about 10 seconds.
9. The method according to any of the preceding clauses, wherein the cooling element is a component of a cooling system selected from the group consisting of: a thermoelectric cooling system, a liquid evaporation cooling system, a solid sublimation cooling systems, a solid melting cooling system, a Joule-Thompson cooling system, a thermodynamic cycle cooling system, an endothermic reaction cooling system and a low-temperature substance cooling system.
10. The method according to Clause 9, wherein the cooling system comprises a thermoelectric cooling system.
11. The method according to any of the preceding clauses, wherein the ocular tissue site has an area ranging from 3 to 60 mm².
12. The method according to Clause 12, wherein the area ranges from 12 to 40 mm².
13. The method according to Clause 11, wherein the ocular tissue site includes at least one area located 2-6 mm from the limbus.
15. The method according to any of the preceding clauses, wherein the method is employed in a method for treating the subject for an ocular disease.
16. The method according to Clause 15, wherein the method further comprises administering a therapeutic agent to the subject via the ocular tissue site.
17. A method of reducing pain experienced by a subject during an ocular procedure, the method comprising:

contacting an ocular tissue site with a cooling element in a manner sufficient to produce anesthesia or analgesia at the ocular tissue site of a subject to reduce pain experienced by the subject during the ocular procedure.
18. The method according to Clause 17, wherein the cooling element has a temperature ranging from −7 to −20° C.
19. The method according to Clause 17, wherein the cooling element has a temperature ranging from −8 to −15° C.
20. The method according to Clause 17, wherein the cooling element has a temperature of about −10° C.
21. The method according to Clause 17, wherein the cooling element has a temperature of about −7° C.
22. The method according to Clause 17, wherein the cooling element has a temperature of about −5° C.
23. The method according to any of Clauses 17 to 22, wherein the application time ranges from 5 to 30 seconds.
24. The method according to Clause 23, wherein the application time ranges from 10 to 20 seconds.
25. The method according to Clause 17, wherein the cooling element has a temperature of about −10° C. and the application time is about 10 seconds.
26. The method according to any of Clauses 17 to 25, wherein the cooling element is a component of a cooling system selected from the group consisting of: a thermoelectric cooling system, a liquid evaporation cooling system, a solid sublimation cooling systems, a solid melting cooling system, a Joule-Thompson cooling system, a thermodynamic cycle cooling system, an endothermic reaction cooling system and a low-temperature substance cooling system.
27. The method according to Clause 26, wherein the cooling system comprises a thermoelectric cooling system.
28. The method according to any of Clauses 17 to 27, wherein the ocular tissue site has an area ranging from 3 to 60 mm².
29. The method according to Clause 28, wherein the area ranges from 12 to 40 mm².
30. The method according to Clause 29, wherein the ocular tissue site includes at least one area located 2-6 mm from the limbus.
31. The method according to any of Clauses 17 to 27, wherein the method is employed in a method for treating the subject for an ocular disease.
32. The method according to Clause 31, wherein the procedure comprises administering a therapeutic agent to the subject via the ocular tissue site.
33. A method of reducing pain experienced by a subject after an ocular procedure, the method comprising:

contacting an ocular tissue site with a cooling element in a manner sufficient to produce anesthesia or analgesia at the ocular tissue site of a subject to reduce pain experienced by the subject after the ocular procedure.
34. The method according to Clause 33, wherein the cooling element has a temperature ranging from −7 to −20° C.
35. The method according to Clause 33, wherein the cooling element has a temperature ranging from −8 to −15° C.
36. The method according to Clause 33, wherein the cooling element has a temperature of about −10° C.
37. The method according to Clause 33, wherein the cooling element has a temperature of about −7° C.
38. The method according to Clause 33, wherein the cooling element has a temperature of about —5° C.
39. The method according to any of Clauses 33 to 38, wherein the application time ranges from 5 to 30 seconds.
40. The method according to Clause 39, wherein the application time ranges from 10 to 20 seconds.
41. The method according to Clause 33, wherein the cooling element has a temperature of about −10° C. and the application time is about 10 seconds.
42. The method according to any of Clauses 33 to 41, wherein the cooling element is a component of a cooling system selected from the group consisting of: a thermoelectric cooling system, a liquid evaporation cooling system, a solid sublimation cooling systems, a solid melting cooling system, a Joule-Thompson cooling system, a thermodynamic cycle cooling system, an endothermic reaction cooling system and a low-temperature substance cooling system.
43. The method according to Clause 42, wherein the cooling system comprises a thermoelectric cooling system.
44. The method according to any of Clauses 33 to 43, wherein the ocular tissue site has an area ranging from 3 to 60 mm².
45. The method according to Clause 44, wherein the area ranges from 12 to 40 mm².
46. The method according to Clause 45, wherein the ocular tissue site includes at least one area located 2-6 mm from the limbus.
47. The method according to any of Clauses 33 to 46, wherein the method is employed in a method for treating the subject for an ocular disease.
48. The method according to Clause 47, wherein the procedure comprises administering a therapeutic agent to the subject via the ocular tissue site.
49. A device for producing anesthesia or analgesia at an ocular tissue site, the device comprising:

a cooling system having a target tissue contacting cooling element configured to have a temperature ranging from 0 to −30° C. for an application time of 30 seconds or less.
50. The device according to Clause 49, wherein the cooling element is configured to have a temperature ranging from −5 to −20° C.
51. The device according to Clause 49, wherein the cooling element is configured to have a temperature of about −10° C.
52. The device according to Clause 49, wherein the cooling element has a temperature of about −7° C.
53. The device according to Clause 49, wherein the cooling element has a temperature of about −5° C.
54. The device according to any of Clauses 49 to 53, wherein the application time ranges from 5 to 30 seconds.
55. The device according to Clause 54, wherein the application time ranges from 10 to 20 seconds.
56. The device according to Clause 49, wherein the cooling element is configured to have a temperature of about −10° C. for an application time of about 10 seconds.
57. The device according to any of Clauses 49 to 56, wherein the cooling system is selected from the group consisting of: a thermoelectric cooling system, a liquid evaporation cooling system, a solid sublimation cooling systems, a solid melting cooling system, a Joule-Thompson cooling system, a thermodynamic cycle cooling system, an endothermic reaction cooling system and a low-temperature substance cooling system.
58. The device according to Clause 57, wherein the cooling system comprises a thermoelectric cooling system.
59. The device according to any of Clauses 49 to 58, wherein the cooling element includes an ocular tissue contacting surface having an area ranging from 3 to 60 mm².
60. The device according to Clause 49, wherein the area ranges from 12 to 40 mm².
61. The device according to any of Clauses 49 to 60, wherein the device is configured to be employed in a method of treating a subject for an ocular disease.

All publications and patent applications cited in this specification are herein incorporated by reference as if each individual publication or patent application were specifically and individually indicated to be incorporated by reference. The citation of any publication is for its disclosure prior to the filing date and should not be construed as an admission that the present invention is not entitled to antedate such publication by virtue of prior invention.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

1. A device for producing anesthesia or analgesia at an ocular tissue site, the device comprising:
a cooling system having a target tissue contacting cooling element configured to have a temperature ranging from 0 to −30° C. for an application time of 30 seconds or less.

2. The device according to claim 2, wherein the cooling element is configured to have a temperature ranging from −5 to −20° C.

3. The device according to claim 1, wherein the application time ranges from 5 to 30 seconds.

4. The device according to claim 3, wherein the application time ranges from 10 to 20 seconds.

5. The device according to claim 1, wherein the cooling element is configured to have a temperature of about −10° C. for an application time of about 10 seconds.

6. The device according to 5 claim 1, wherein the cooling system is selected from the group consisting of: a thermoelectric cooling system, a liquid evaporation cooling system, a solid sublimation cooling systems, a solid melting cooling system, a Joule-Thompson cooling system, a thermodynamic cycle cooling system, an endothermic reaction cooling system and a low-temperature substance cooling system.

7. The device according to claim 6, wherein the cooling system comprises a thermoelectric cooling system.

8. The device according to claim 1, wherein the cooling element includes an ocular tissue contacting surface having an area ranging from 3 to 60 mm².

9. The device according to claim 8, wherein the area ranges from 12 to 40 mm².

10. The device according to claim 1, wherein the device is configured to be employed in a method of treating a subject for an ocular disease.

11. A method of producing anesthesia or analgesia at an ocular tissue site of a subject, the method comprising:
contacting the ocular tissue site with a cooling element having a temperature ranging from 0 to −30° C. for an application time of 30 seconds or less so as to produce anesthesia or analgesia at the ocular tissue site.

12. (canceled)

13. The method according to claim 11, wherein the method is employed in a method for treating the subject for an ocular disease.

14. The method according to claim 13, wherein the method further comprises administering a therapeutic agent to the subject via the ocular tissue site.

15. The method according to claim 11, wherein the subject is a human.
